SEQUENCE LISTING (1 sequence)

SEQ ID NO: 1 - DNA, 1530 bp, Pseudomonas stutzeri
  misc_feature (68)..(68): n is a, c, g, or t
tttgatcctggctcagattgaacgctggcggcaggcctaacacatgcaagtcgaacggtaacaggaancagcttgctgnttcgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaaaggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggctggatcacctcctt

(19) United States
(12) Patent Application Publication
(10) Pub. No.: US 2012/0289456 A1
(43) Pub. Date: Nov. 15, 2012

(54) ANTIMICROBIAL PROTEIN COMPOSITIONS AND USES THEREOF

(75) Inventors: MICHAEL J. P. LAWMAN, Tampa, FL (US); Selena Savell, San Diego, CA (US); Shabnam Eidizadeh, Lutz, FL (US)

(73) Assignee: ST. JOSEPH'S CHILDREN'S HOSPITAL, TAMPA, FL (US)

(21) Appl. No.: 13/370,344

(22) Filed: Feb. 10, 2012

Related U.S. Application Data
(63) Continuation-in-part of application No. 13/060,448, filed on Feb. 24, 2011, now Pat. No. 8,114,657.
(60) Provisional application No. 61/159,128, filed on Mar. 11, 2009.
(60) Provisional application No. 61/091,535, filed on Aug. 25, 2008.

(51) Int. Cl. A61K 38/02; A61P 31/04; C12N 1/20

(52) U.S. Cl. 514/2.7; 514/2.4; 514/2.8; 435/252.1

(57) ABSTRACT

Several bacterial species were isolated from marine segment obtained from seabed sediment at depths exceeding 1700 feet. At least four of the bacteria produced a compound that showed antibacterial activity against one or more multiple-drug-resistant (MDR) bacteria isolated from hospitals and clinics. One isolate, SJCH-12, exhibited a broad range of activity against MDR strains tested, including methicillin resistant Staphylococcus aureus (MRSA).

### CROSS REFERENCE TO RELATED APPLICATIONS

This application is a continuation-in-part of U.S. Ser. No. 13/060,448, filed Feb. 24, 2011, now U.S. Pat. No. 8,114,657, which is the U.S. national stage application of International Patent Application No. PCT/US2009/054766, filed Aug. 24, 2009, which claims the benefit of U.S. Provisional Application Ser. No. 61/091,535, filed Aug. 25, 2008, and Ser. No. 61/159,128, filed Mar. 11, 2009, the disclosures of which are hereby incorporated by reference in their entirety, including all figures, tables and amino acid or nucleic acid sequences.

The Sequence Listing for this application is labeled “Seq-List.txt” which was created on Aug. 24, 2009 and is 3 KB. The entire contents of the sequence listing is incorporated herein by reference in its entirety.

### BACKGROUND OF THE INVENTION

1. Field of the Invention

The invention relates generally to polypeptide antibiotics produced by bacteria isolated from marine environments. In particular, the present invention is directed to antibacterial compositions useful for treating a broad spectrum of bacterial infections.

2. Description of Background Art

The search for new and advanced cancer treatments is dependent upon the discovery of new compounds, the development of new therapeutic strategies and advances in predictive models for disease. Due to the immense technical advances that have been made in the pharmaceutical industry and medicine, there is a resurging interest in the use of natural products in the formulation of therapeutic drugs. In fact, many of the drugs in use today are derivatives of natural products, which provide additional incentive to take further advantage of the biodiversity available for the discovery of new drugs, particularly in the area of cancer therapy.

In response to evolutionary pressures imposed over time, new molecules and compounds constantly evolve, resulting in a structural diversity against which modern technologies such as combinatorial chemistry cannot compete. To make use of the biological and chemical diversity of natural products, it has become increasingly clear that the most powerful approach in the search for new drugs begins with drug leads revealed by natural product-based drug discovery techniques, and subsequently utilizing genomic—based platforms to identify and produce the lead compounds that are the basis for next generation drugs.

Since the discovery of penicillin in 1929, nearly 50,000 natural products have been isolated from microorganisms. Over 10,000 of these compounds have been shown to have biological activity and 100 of these are in use today in the treatment of a wide range of human and animal diseases. Numerous antibiotics and anticancer agents have been identified and have provided a powerful weapon in the arsenal of drugs for treating infectious diseases.

Microbes, unfortunately, constantly adapt to changing environments so that multiple drug resistance develops rapidly after infectious microorganisms are exposed to new antimicrobial agents. This resistance poses a continuing challenge to identify new agents that will effectively control bacterial growth and propagation.

A majority of antibiotics have, like penicillin, been isolated from natural sources or derived from bioactive natural products. The list is extensive and includes β-lactam antibiotics such as the cephalosporin family, chloramphenicol, vancomycin, bacitracin and structurally diverse compounds such as brominated pyrroles, magnesidins, and substituted biphenyldiols. The sources of these compounds are equally diverse and range from soil bacteria to marine pseudomonads and bioflora.

The oceans of the world cover over 70% of the earth's surface and have been described as being the “mother of the origin of life.” Given the uniqueness of the environment found in the oceans at various geographic locations around the world, organisms have responded by developing the structurally unique natural compounds required for adaptation and survival in a marine environment. Many of these compounds show pharmacologic activity against many human illnesses, ranging from infectious diseases to cancers. Previously discovered life saving drugs and potentially new drugs have been and are being isolated from microorganisms, algae, plants and invertebrates. With the advent of the technological advances made in the biotechnology, biomedical and pharmaceutical arenas, the discovery of new therapeutics from aquatic organisms has become a “new science”. Of the 25,000 plant species classified to date, only 10% have been studied in attempts to discover new therapeutically active compounds.

Marine environments have been even less utilized as sources of new drugs. Over 80% of the world's plant and animal species are found in a marine environment. Of the 34 fundamental phyla representing life, 17 occur on land whereas 32 occur in the ocean. As of 2004, basic research has led to the isolation of approximately 14,000 marine natural products with approximately 10-15 different natural products entering clinical testing (FDA sponsored clinical trials) in the cancer, infectious disease, pain and inflammatory disease fields.

Despite recognition that the marine environment is an exceptional reservoir of bioactive natural products arising from an amazing diversity of life, the identification of potential new drugs from the oceans has progressed only slowly. Bioactive compounds have been extracted from a variety of marine organisms: tunicates, sponges, soft corals, sea hares, nudibranchs, bryozoans, sea slugs and microorganisms. Spongouridine and spongothymidine from the Caribbean sponge were among the first bioactive compounds isolated over fifty years ago.

Drug research studies on sponge-derived products has led to the development of anticancer and antiviral compounds. Two successfully launched marine organism-derived (or analog derived) products reaching the clinics within the last 30 years are Acyclovir (synthetically known as Ara A) and cephalosporin. Synthetic Ara A was modeled on the previously isolated sponge-derived spongothymidine or spongouridine and later isolated as a natural product from Eunicella cavolini. The antibiotic mimosamycin was isolated from a nudibranch sea slug and also found in certain sponges.

Secondary metabolites of marine organisms have also been studied over the past decades, which have often exhibited unique structures. Between 2000 and 2005, ziconotide, aplidine, KRN7000, discodermolide, bryostatin, synthadotin, dolastatin 10, oblidotin, halichondrin, HTI-286, kahalalide F, spisulosine, squalamine and 743 have been identified from marine sources as potential drug candidates (Butler, 2005; Newman and Cragg, 2004A; Newman and Cragg, 2004B.) Several of these compounds are or have been in clinical trials.

Ziconotide, a 24-27 amino acid peptide from the -conotoxin cyclic cysteine known family was identified from cone snail (Conus magnus) venom. It is a novel non-opioid analgesic that blocks the N-type voltage gated channel and was developed for management of severe chronic pain.

Aplidine is an analog of the didemnins isolated from Aplidium albicans, a Mediterranean tunicate, and is reported to show activity against medullary thyroid carcinoma, renal carcinoma, melanoma and tumors of neuroendocrine origin and to inhibit secretion of vascular endothelial growth factor (VEGF) (Taraboletti, 2004).

Agelasphins are new glycosphingolipids isolated as antitumor agents from Agelas mauritianus, an Okinawan sponge. KRN7000 is a synthetic derivative in clinical trials whose activity is attributed to natural killer cell activation effected as a ligand of VαT cell antigen receptor (Hayakawa, et al., 2003).

Bryostatin was isolated from Bugula neritina and binds to the same receptors as phorbol esters but differs in not having any tumor promoting activity. Binding of bryostatin downregulates protein kinase C isoforms in several tumor cells, causing inhibition of growth, alteration of differentiation and/or cell death (Newman, 2005).

Discodermolide has been isolated from Discodermia dissolute and found to inhibit tumor cell growth in vitro (Capon, 2001) as do dolastatins, which are linear peptides isolated from the Indian Ocean sea hare Dolabela auricularia (Pettit, et al., 1989).

Other potential drugs have been isolated from marine sources, some of which are in or are candidates for clinical trial studies. Table 1 is a list of examples of additional compounds recovered from marine environments and the organism from which it was isolated

Secondary metabolites of marine organisms have also been studied over the past decades, which have often exhibited unique structures. Between 2000 and 2005, ziconotide, aplidine, KRN7000, discodermolide, bryostatin, synthadotin, dolastatin 10, soblidotin, halichondrin, HTI-286, kahalalide F, spisulosine, squalamine and ecteinascidin 743 have been identified from marine sources as potential drug candidates (Butler, 2005; Newman and. Cragg, 2004A; Newman and Cragg, 2004B.) Several of these compounds are or have been in clinical trials.

Ziconotide, a 24-27 amino acid peptide from the -conotoxin cyclic cysteine know family was identified from cone snail (Conus magnus) venom. It is a novel non-opioid analgesic that blocks the N-type voltage gated channel and was developed for management of severe chronic pain.

TABLE 1

| DRUG | SOURCE | ORGANISM |
|---|---|---|
| Halichondrin E7389 | | Halichondria okadai |
| HTI-286 | sponge | Hemiasterella minor |
| Kahalalide F | mollusk | Elysia rufescens |
| Spisulosine | | Spisula polynyma |
| Squalamine | dogfish shark | Squalus acanthias |
| Ecteinascidin | marine tunicate | Ecteinascidia turbinate |

The vast majority of compounds currently in clinical trials or being considered as potential drug candidates exhibit antitumor activity, although the search for other classes of drugs has currently produced far fewer candidates.

Marine microorganisms have produced several potential antimicrobials, and new antibiotics isolated over the past several years, include lololatin, agrochelin (Acebal, et al., 1999) and sesbanimides from agrobacterium, pelagiomicins from Pelagiobacter variabilis, d-indomycinone from a Streptomyces sp. (Biabini, et al., 1997) and dihydrophencomycin from Streptomyces (Pusecker, et al., 1997). Alteromonas has also been reported to produce antibiotics and other bioactive substances (Gauthier, et al., 1995).

Marine sources historically have been underutilized in the search for new drugs and are only now being more fully exploited by interdisciplinary groups devoted solely to drug discovery research. Despite some progress in identifying new antimicrobial compounds, there are a limited number of marine-derived compounds that are active against MDR bacteria. In 2005, only 6 new anti-bacterial pharmaceuticals were reported to be in the development pipeline (Usdin, 2006).

Recently, an unusual pair of antibiotics isolated from bacteria obtained from ocean sediments have been identified by Fenical, et al. (2008). The new compounds have a basic pyrrole structure that is an N, C2-linked bispyrrole, and exhibit antimicrobial activity against methicillin-resistant S. aureus.

The rapid increase in the number of MDR strains and the decreasing effectiveness of currently used antimicrobials, are strong indications of the need for new and effective first-generation antibiotics.

### SUMMARY OF THE INVENTION

The present invention provides antibacterial compositions based on novel compounds obtained from marine sources. Several different antibiotic activities were identified in samples provided from core seabed samples on the bottom of the Atlantic Ocean. The samples were retrieved near the shipwreck of the SS Republic lost in a hurricane on Oct. 25, 1865, which sank approximately 100 miles off the Georgia coast and was found nearly a century later on the seabed at approximately 518 meters depth.

The active compounds produced by the isolated marine bacteria are relatively small proteins with molecular weights in the range of 5 kDa. They appear to be produced by several species of bacteria.

Thirteen bacterial isolates were isolated from the marine sediment samples of which four had antibacterial activity (SJCH-3, 10, 11 and 12); however, only SJCH-12 exhibited a broad range of antibacterial activity and only SJCH-12 had high activity against MRSA

Bacterial isolate SJCH-12 was found to comprise two bacteria, one of which appeared to be a pseudomonad, tentatively identified as a Pseudomonas stutzeri species while the other, also a gram negative rod was similar to Shigella or an E. coli genus. The antibacterial polypeptide produced by the SJCH-12 culture may have required the presence of both bacteria but it was not determined whether or not actually produced by a single species.

### BRIEF DESCRIPTION OF THE FIGURES

FIG. 1 illustrates the overall process used for isolating the marine microorganisms.

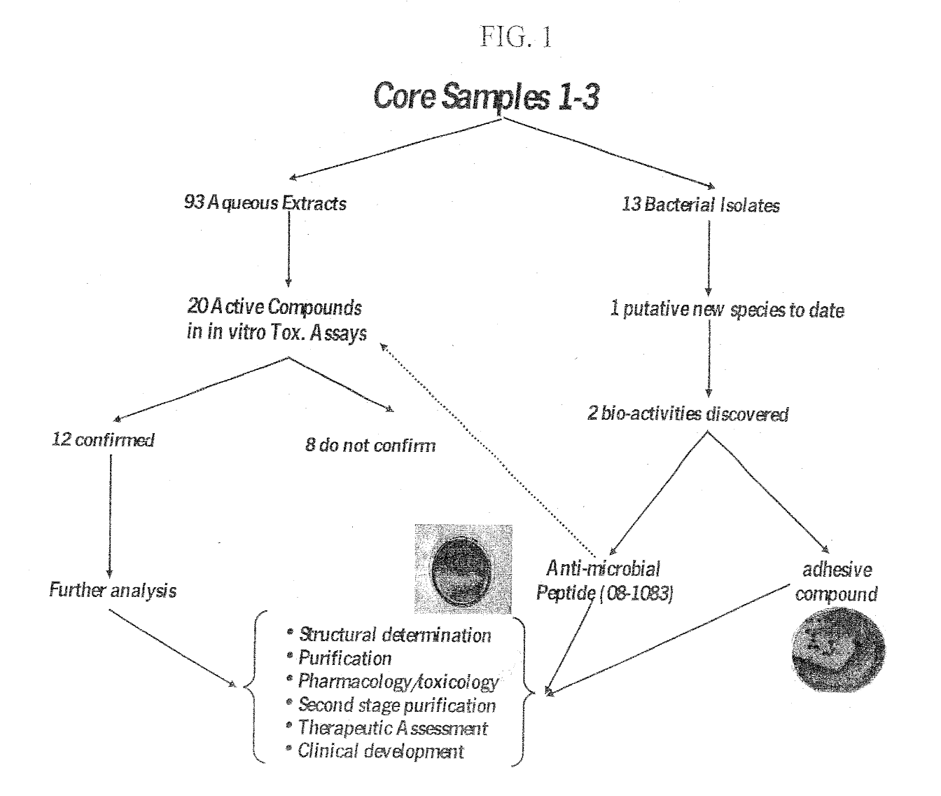

FIG. 2 shows the antibiotic activity of protein 08-1083 in the isolate from marine organism SJCH-12 separated by SDS-gel electrophoresis of soluble SJCH-12 proteins.

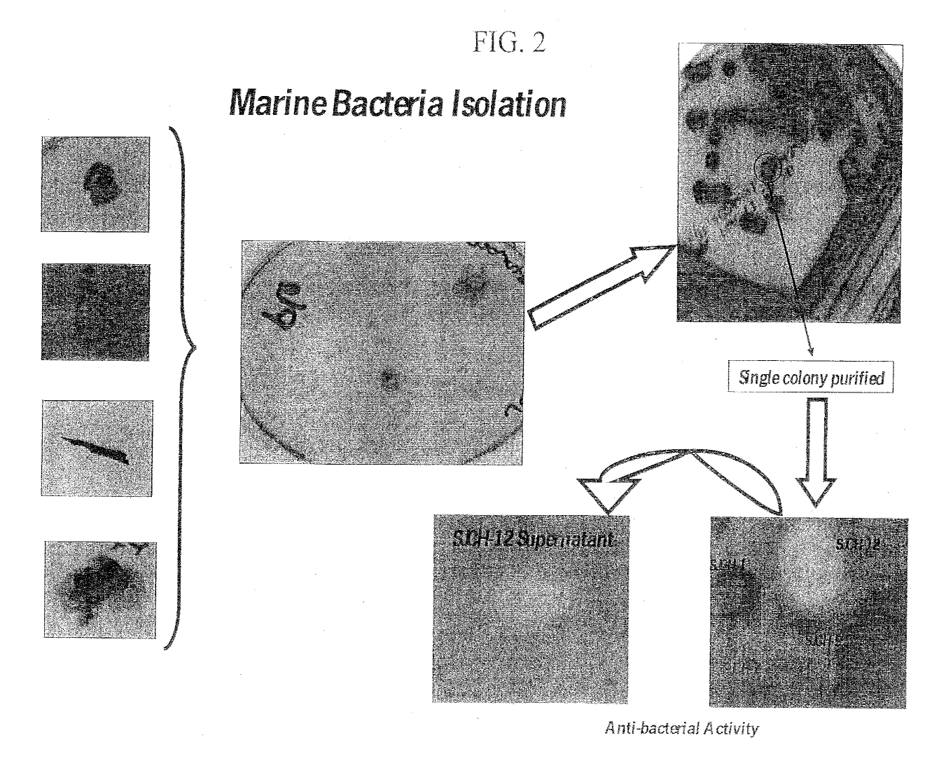

FIG. 3 is a PAGE gel electrophoresis demonstrating the protein profile of the antibiotically active agent in bacterial lysate SJCH-12 on a 16% acrylamide gel. The location of spots G and H having respective measured pis of 5.9 and 6.8-7.2 is shown in FIG. 4.

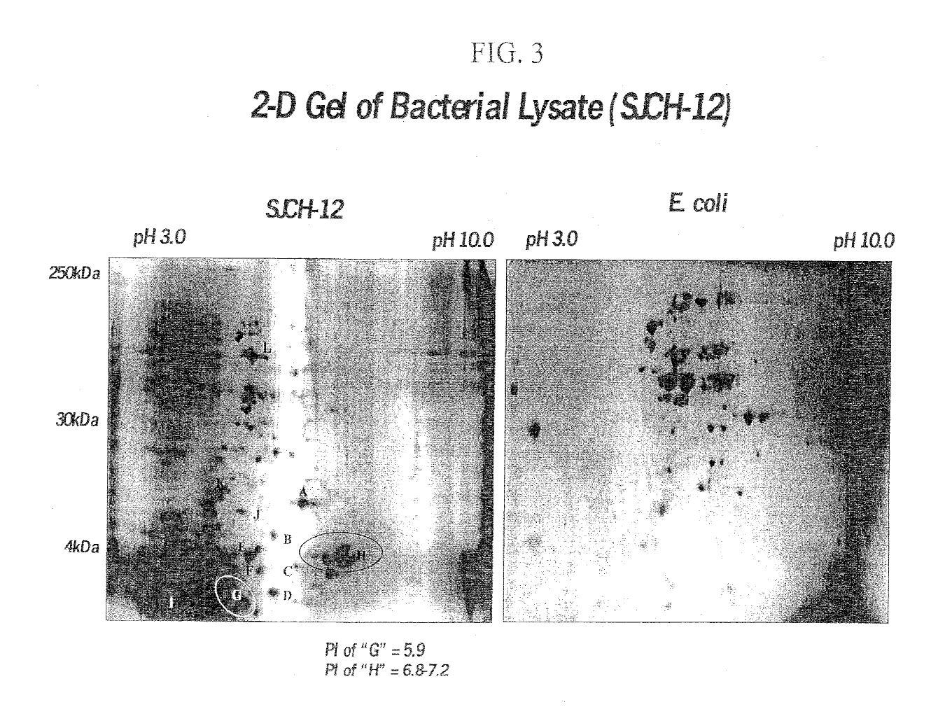

FIG. 4 is a two dimensional gel electrophoresis of the SJCH-12 bacterial lysate. Zone H is shown in FIG. 3 as the corresponding spot with a pI of 6.8-7.2. Zone G is shown in FIG. 3 as having a measured pI of 5.9.

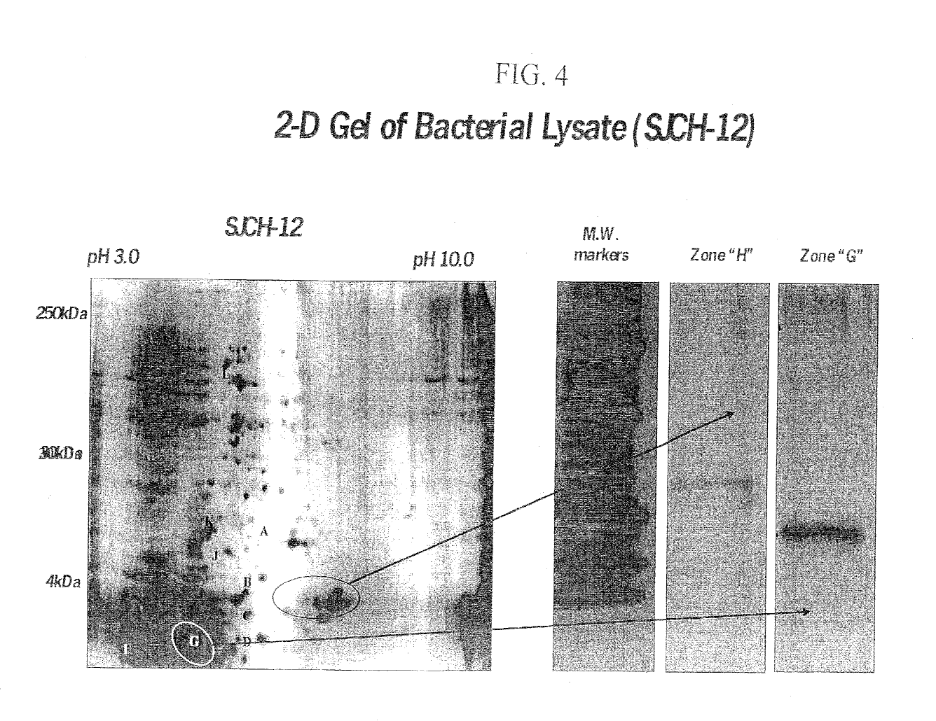

FIG. 5 is a photograph of SJCH-12 bacterium showing the rod shape and the 16SrRNA gene sequence (SEQ ID NO:1) of strain SJCH-12.

FIG. 6 shows the activity of partially purified protein 08-1083 from bacteria SJCH-12 against MRSA and E. coli.

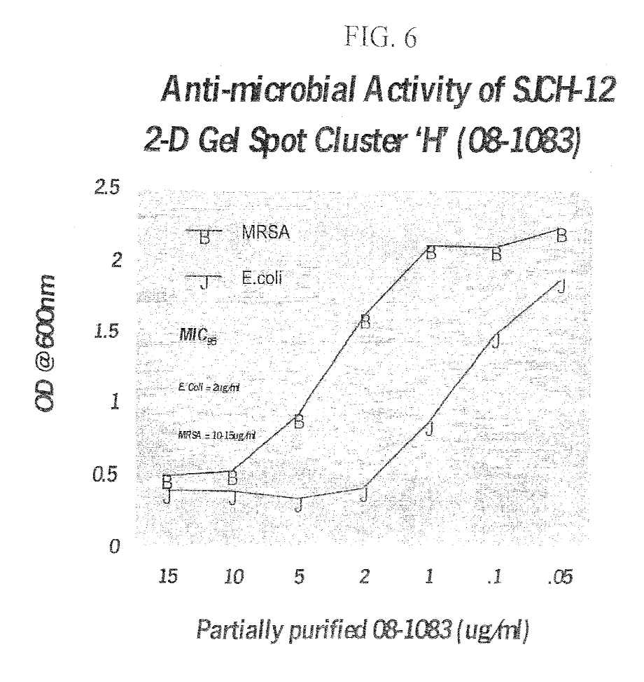

FIG. 7 shows activity of partially purified protein 08-1083 against MRSA compared with amoxicillin.

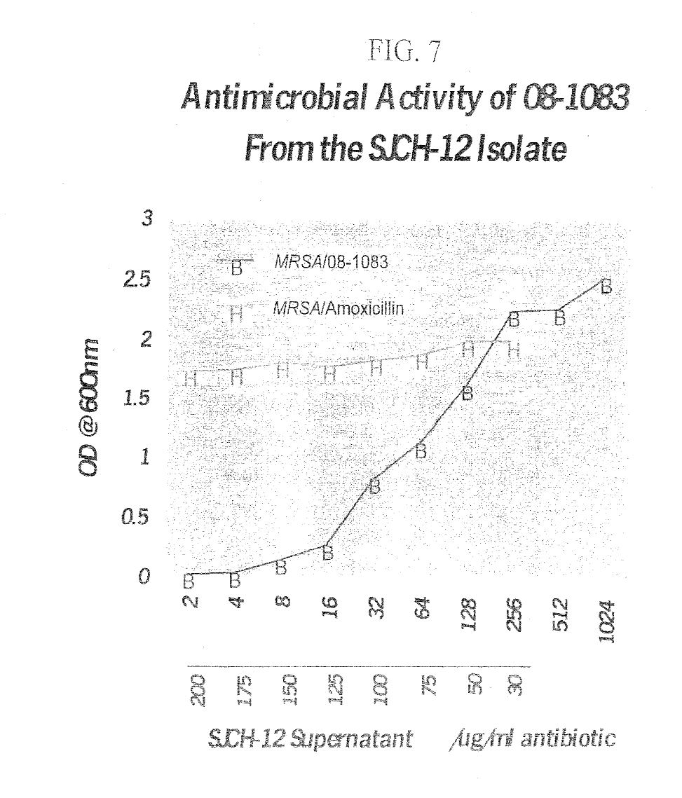

FIG. 8 shows activity of partially purified protein 08-1083 against E. coli compared with ampicillin.

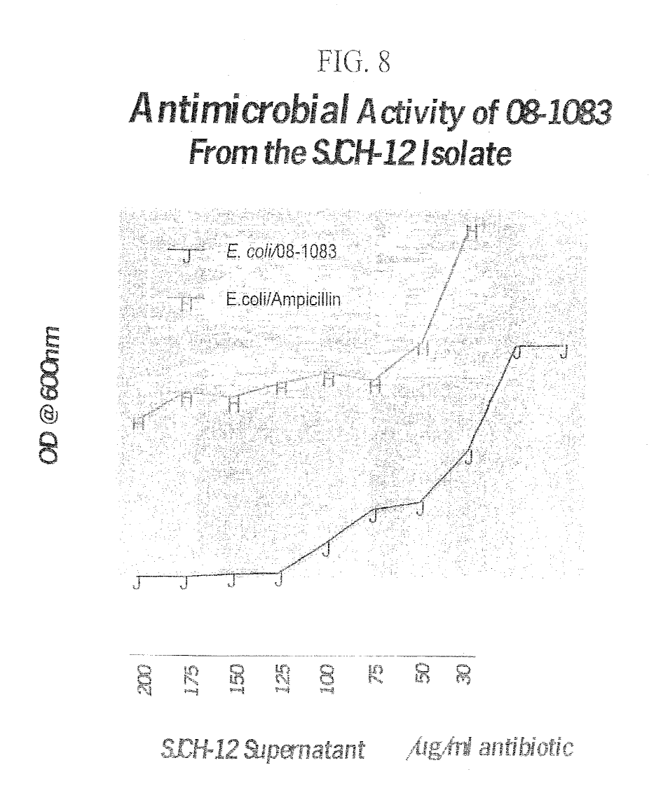

### DETAILED DESCRIPTION OF THE INVENTION

The present invention provides novel polypeptide compositions that show a wide range of antibacterial activity. The active compounds were isolated from marine bacteria isolated from core sample sediments which included coarse brown sediment with worm casings visible (core sample 1); rocky with grey sediment (core sample 2); and grey sediment containing rock-like material (core sample 3). Bacterial colonies were isolated from these samples. Of 13 bacterial colonies, at least 1 putative new species of bacterium was isolated and 2 bio-activities identified, one of which was a highly antimicrobial peptide identified as 08-1083 and the other an unidentified compound with adhesive properties. Of 93 core sample extracts, 20 active compounds were indicated in in vitro Tox. A Assays.

Eleven samples were taken from the three core samples and plated on LB agar or grown in LB broth. Thirteen bacterial isolates were colony purified and identified as SJCH 1-13. Of the 13 isolates, 4 exhibited antimicrobial activity. SJCH-12 isolate showed activity against 8 of 11 organisms tested, while SJCH-11 exhibited activity against 3 of the organisms; SJCH-10 against two; and SJCH-3 against only one, see Table 2.

Pharmaceutical compositions containing one or more of the antibiotics produced by any of the described isolates are preferably administered parenterally, intraperitoneally, intradermally or intramuscularly. Pharmaceutical forms suitable for injection include sterile aqueous solutions or dispersions for extemporaneous preparation of the solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained by the use of a coating such as lecithin, by the maintenance of the required particle size in case of a dispersion and by the use of surfactants. The prevention of the action of microorganisms can be effected by various antibacterial and antifungal agents such as parabens, chlorobutanol, phenol, sorbic acid, thimerosal and the like. In many cases, isotonic agents may be included, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Such compositions can be prepared as injectables, either as liquid solutions or suspensions; solid forms suitable for solution in, or suspension in, liquid prior to injection can also be prepared. The preparation can also be emulsified.

Upon formulation, solutions or solid forms will be administered in a manner compatible with the dosage formulation and in such amount as is therapeutically effective. Such determinations are routinely determined by those skilled in the art, by testing for toxicity (LD₅₀ for example) and amounts sufficient to produce a therapeutic effect. The formulations can be administered in a variety of dosage forms.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intradermal and intraperitoneal administration. In this connection, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Oral administration of drugs is preferable to injectable forms but may be an issue with protein composition because of metabolism in the gut. Oral forms absorbed in the small intestine may be degraded in the stomach and therefore not taken into the body. Stabilization of tablet forms of proteins sensitive to acid decomposition may be achieved by coating the granulated drug with a fat or wax, either simultaneously or step-wise with a polymer coating such as polyvinyl alcohol. This is expected to provide added stability toward gastrointestinal absorption. This also would address individual differences in patients with different gastric acidity. Lipid materials have been used in this manner in formulations of anti-parasitic compounds. Examples of lipids suitable for co-coating are found in Application Serial No. 2006/0068020 and Application Serial No. 2006/0067954. In choosing a lipid such as a fat or wax, biocompatibility is a factor as is solubilization. For example, long chain fatty acids can be dissolved in alcohol with polyvinyl alcohol and then used to coat a highly granulated drug before drying and compressing.

### EXAMPLES

Materials and Methods

Marine seabed samples were obtained from an Odyssey Dive Site located in the Atlantic ocean approximately 100 miles cast of the Georgia coastline. Samples comprised seabed material recovered by Odyssey Marine Exploration (Tampa, Fla.) remote operated vehicle during routine dive operations at 1700 feet. Samples were stored at 4° C. or ambient temperature prior to analysis.

Bacteria isolate SJCH-12 was deposited on Oct. 20, 2008 at the Deutsche Sammlung von Mikroorganismen und Zellkulturen GmbH, Inhoffenstrasse 7B, 38124 Braunschweig, Germany under the conditions of the Budapest Treaty and assigned accession number DSM 21971. The deposit was made under the terms of the Budapest Treaty and all restrictions imposed by the depositor on the availability to the public of the deposited material will be irrevocably removed upon the granting of a patent in compliance with 37 CFR 1.801-37CFR 1.809.

Bacterial cultures were grown and isolated under standard conditions on LB agar or in LB broth. 11 samples were examined from 3 core samples before plating on LB agar or placed in LB broth.

The following examples are provided as illustrations of the invention and are in no way to be considered limiting.

### Example 1

### Isolation of Bacterial Colonies

A total of 13 isolates were obtained. These are listed in Table 2. Of these, isolates SJCH-10, SJCH-11, SJCh-12 and SJCH-13 exhibited antibacterial activity of varying degrees against one or more of the multi-drug resistant (MDR) bacterial strains, K. pneumoniae, K. Oxytoca and A. baumannii as shown in Table 3 in Example 2.

TABLE 2
STRAIN

Aeromonas hydrophila
Staphylococcus aureus
Escherichia coli
Enterobacter aerogenes
Proteus vulgaris
Klebsiella pneumonia
Providencia alcalifaciens
Klebsiella oxytoca
Acinetobacter baumannii
Serratia liquefacien
Pseudomonas aeruginosa

### Example 2

### Antibacterial Activity of 13 Bacteria Isolates

Isolates selected from the bacterial colonies in Example 1 were tested for antibacterial activity against several strains of bacteria listed in Table 2 and isolated from hospital surgical suites and patient wards, including multi-drug resistant (MDR) strains. The MDR strains are Klebsiella pneumoniae (KP), Klebsiella oxytoca (KO) and Acinetobacter baumannii (AB). Antibacterial activities are shown in Table 3.

TABLE 3
Antibiotic Sensitivity Assay
Marine Bacterial Isolates

| Bacterial | | | | | | | | | | | |
|---|---|---|---|---|---|---|---|---|---|---|---|
| Strains | SJCH-1 | SJCH-2 | SJCH-3 | SJCH-5 | SJCH-7 | SJCH-8 | SJCH-9 | SJCH-10 | SJCH-11 | SJCH-12 | SJCH-13 |
| AH | Neg | Neg | Neg | Neg | Neg | Neg | Neg | Neg | Neg | Neg | Neg |
| MRSA | Neg | Neg | Neg | Neg | Neg | Neg | Neg | Neg | +/− | Neg | Neg |
| EC | Neg | Neg | Neg | Neg | Neg | Neg | Neg | Neg | Neg | + | Neg |
| EA | Neg | Neg | Neg | Neg | Neg | Neg | Neg | Neg | Neg | + | Neg |
| PV | Neg | Neg | Neg | Neg | Neg | Neg | Neg | Neg | Neg | + | Neg |
| KP | Neg | Neg | + | Neg | Neg | Neg | Neg | + | + | + | Neg |
| PA | Neg | Neg | Neg | Neg | Neg | Neg | Neg | Neg | Neg | Neg | Neg |
| KO | Neg | Neg | Neg | Neg | Neg | Neg | Neg | + | + | + | Neg |
| AB | Neg | Neg | Neg | Neg | Neg | Neg | Neg | Neg | Neg | + | +/− |
| SL | Neg | Neg | Neg | Neg | Neg | Neg | Neg | Neg | Neg | Neg | Neg |
| PSA | Neg | Neg | Neg | Neg | Neg | Neg | Neg | Neg | Neg | + | Neg |

AH = Aeromonas hydrophila
SA = Sytaphylococcus aureus
EC = E. coli
EA = Enterobacter aerogenes
PV = Proteus vulgaris
KP = Klebsiella pneumoniae
PA = Providencia alcalifaciens
KO = Klebsiella oxytoca
AB = Acinetobacter baumannii
SL = Serratia liquefaciens
PSA = Pseudomonas aeruginosa

### Example 3

The bacteria from which antibacterially active 08-1083 was isolated were highly adherent when cultured on agar plates. No determination was made as to whether this was an immobilization phenomenon or the result of chemical bonding. Agar is a polymannuronic acid formed as a polymer of agarobiose, composed of disaccharide units of D-galactose and 3,6-anhydro-L-galactose. Agar is commonly used as a cell immobilization medium which allows trapped cells to grow while entrapped in a microporous membrane.

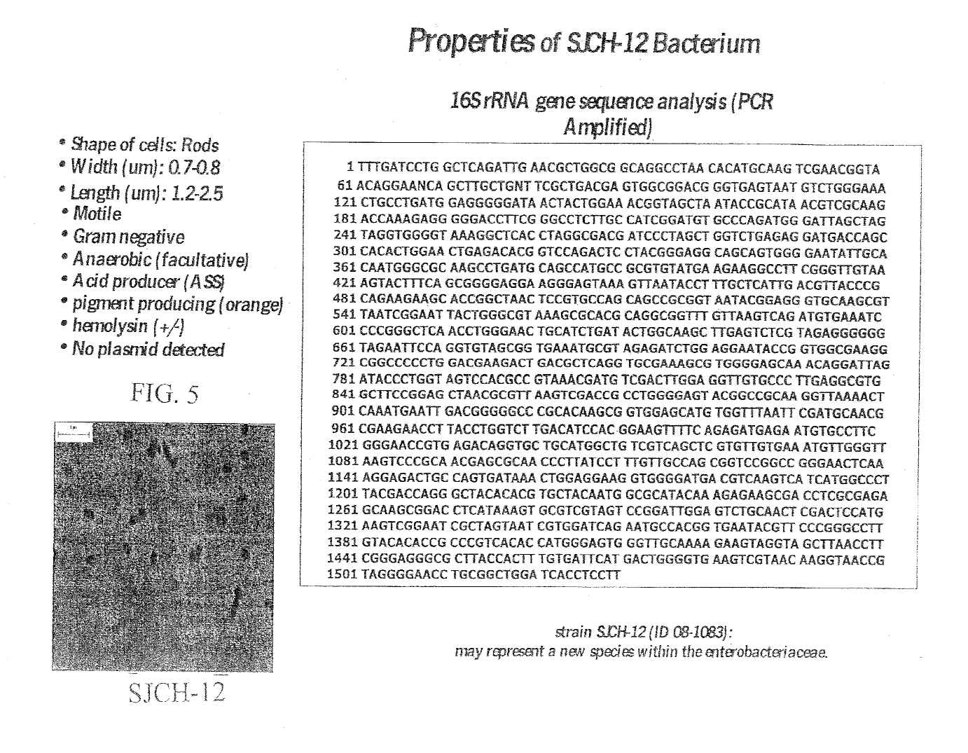

The SJCH-12 bacteria appeared as Gram negative rods with a width of 0.7-0.8 microns and a length of 1.2-2.5 microns. They are anaerobic (facultative), acid producers (ASS), pigment producing (orange). No plasmid was detected and they are +/− with respect to hemolysin. FIG. 5 is a slide showing the rod shape.

The bacterial SJCH-12 (08-1083) cultures are highly stable at 4°, 25°, and 37° C. for at least 30 days and at 4° C. for at least 6 months. There is some loss of stability at 25° and 37° C. on 6 month storage. Production is increased after 6 months compared to 30 days while secreted protein remains good up to at least 6 months.

### Example 4

### 16S rRNA Gene Sequence of 08-1083 Bacterial

Approximately 95% of the 16S rRNA gene sequence of the 08-1083 (SJCH-12) bacteria was determined by direct sequencing of PCR-amplified 16S rDNA, SEQ ID NO:l.

Genomic DNA extraction of bacterial strain SJCH-12 was followed by PCR mediated amplification of the 16S rDNA and purification of the PCR product as described by Rainey, et al. (1996). Purified PCR product was sequenced using the CEQTMDTCS-Quick Start Kit (Beckmann coulter) as directed in the manufacturer's protocol. Sequence reactions were electrophoresed using the CEQtm 8000 Genetic Analysis System.

Sequence data was put into the alignment editor ae2 and aligned manually. It was then compared with the 16S rRNA gene sequences of representative organisms belonging to the Enterobacteriaceae class.

Table 4 is a similarity matrix for SJCH-12 and a phylogenetic tree. The 16S rRNA gene similarity values were calculated by pairwise comparison of the sequences within the alignment. For construction of the phylogenetic dendrogram operations of the ARB package were used (Pruesse, et al., 2007). The phylogenetic tree was constructed by the neighbor-joining method using the correlation of Jukes and Cantor (1969) based on the evolutionary distance values. The roots of the tree were determined by including the 16S rRNA gene sequence of Cronobacter sakazakii into the analysis. The scale bar below the dendrograms indicates the 1 nucleotide substitutions per 100 nucleotides.

The complete 16S rDNA gene sequence of strain SJCH-12 shows a highest similarity of 99.8% (binary value) with Shigella sonnei. On the basis of this result, strain SJCH-12 appears to represent at least one new species within the genus Shigella; however, the genus Escherichia is phylogenetically highly related to this genus; therefore, it cannot be excluded that strain SJCH-12 may also represent a new species within the genus Escherichia.

TABLE 4

| Strains | 1. | 2. | 3. | 4. | 5. | 6. | 7. | 8. | 9. | 10. | 11. |
|---|---|---|---|---|---|---|---|---|---|---|---|
| SJCH-12 (ID 08-1083) | — | | | | | | | | | | |
| Escherichia albertii | 99.2 | — | | | | | | | | | |
| LMG 20976^{T} | | | | | | | | | | | |
| Escherichia coli | 99.6 | 99.2 | — | | | | | | | | |
| ATCC 11775^{T} | | | | | | | | | | | |
| Escherichia fergusonii | 99.8 | 99.2 | 99.6 | — | | | | | | | |
| ATCC 35469^{T} | | | | | | | | | | | |
| Escherichia hermannii | 97.4 | 96.8 | 97.1 | 97.4 | — | | | | | | |
| GTC 347^{T} | | | | | | | | | | | |
| Escherichia vulneris | 99.7 | 99.1 | 99.5 | 99.7 | 97.2 | — | | | | | |
| ATCC 33821^{T} | | | | | | | | | | | |
| Shigella dysenteriae | 98.9 | 98.3 | 98.7 | 98.9 | 97.3 | 98.8 | — | | | | |
| ATCC 13313^{T} | | | | | | | | | | | |
| Shigella flexneri | 99.9 | 99.3 | 99.7 | 99.9 | 97.4 | 99.8 | 99.0 | — | | | |
| ATCC 29903^{T} | | | | | | | | | | | |
| Shigella sonnei | 99.8 | 99.2 | 99.6 | 99.8 | 97.4 | 99.7 | 98.9 | 99.9 | — | | |
| GTC 781^{T} | | | | | | | | | | | |
| Salmonella bongori | 97.3 | 97.1 | 97.1 | 97.3 | 97.7 | 97.1 | 97.9 | 97.4 | 97.3 | — | |
| BR 1859^{T} | | | | | | | | | | | |
| Salmonella enterica | 97.7 | 97.5 | 97.4 | 97.7 | 97.7 | 97.6 | 97.4 | 97.7 | 97.8 | 97.9 | — |
| ssp arizonae | | | | | | | | | | | |
| ATCC 13314^{T} | | | | | | | | | | | |
| Brenneria alni | 93.1 | 92.6 | 92.9 | 93.0 | 93.5 | 92.9 | 93.1 | 93.0 | 93.1 | 93.4 | 93.5 |
| DSM 11811^{T} | | | | | | | | | | | |
| Brenneria salicis | 93.0 | 92.5 | 92.8 | 93.1 | 93.5 | 92.9 | 93.3 | 93.1 | 93.0 | 93.5 | 93.2 |
| DSM 30166^{T} | | | | | | | | | | | |
| Dickeya chrysanthemi | 95.0 | 94.2 | 94.6 | 94.8 | 94.1 | 94.8 | 95.0 | 94.9 | 94.8 | 94.8 | 94.8 |
| DSM 4610^{T} | | | | | | | | | | | |
| Pectobacterium | 95.0 | 94.5 | 95.0 | 95.0 | 95.6 | 94.9 | 95.3 | 95.0 | 95.0 | 95.8 | 95.4 |
| carotovorum | | | | | | | | | | | |
| DSM 30168^{T} | | | | | | | | | | | |
| Edwardsiella hoshinae | 95.0 | 94.4 | 94.7 | 95.0 | 95.0 | 95.0 | 95.3 | 95.0 | 95.0 | 95.0 | 94.2 |
| JCM1679^{T} | | | | | | | | | | | |
| Edwardsiella ictaluri | 95.2 | 94.5 | 94.9 | 95.1 | 95.0 | 95.1 | 95.4 | 95.2 | 95.1 | 95.2 | 94.4 |
| JCM1680^{T} | | | | | | | | | | | |
| Cedecea davisae | 96.5 | 95.9 | 96.2 | 96.5 | 97.7 | 96.5 | 96.4 | 96.5 | 96.5 | 96.2 | 96.2 |
| DSM 4568^{T} | | | | | | | | | | | |
| Cedecea neteri | 96.2 | 95.6 | 95.9 | 96.2 | 97.4 | 96.1 | 96.1 | 96.2 | 96.2 | 96.1 | 96.1 |
| GTC1717^{T} | | | | | | | | | | | |
| Trabulsiella | 95.5 | 94.9 | 95.3 | 95.6 | 96.4 | 95.6 | 96.1 | 95.6 | 95.5 | 96.2 | 96.0 |
| guamensis | | | | | | | | | | | |
| ATCC 49490^{T} | | | | | | | | | | | |
| Cronobacter sakazakii | 96.8 | 96.8 | 96.6 | 96.9 | 96.7 | 96.8 | 97.0 | 96.9 | 96.8 | 96.5 | 96.5 |
| ATCC 29544^{T} | | | | | | | | | | | |

| Strains | 12. | 13. | 14. | 15. | 16. | 17. | 18. | 19. | 20. | 21. |
|---|---|---|---|---|---|---|---|---|---|---|
| SJCH-12 (ID 08-1083) | | | | | | | | | | |
| Escherichia albertii | | | | | | | | | | |
| LMG 20976^{T} | | | | | | | | | | |
| Escherichia coli | | | | | | | | | | |
| ATCC 11775^{T} | | | | | | | | | | |
| Escherichia fergusonii | | | | | | | | | | |
| ATCC 35469^{T} | | | | | | | | | | |
| Escherichia hermannii | | | | | | | | | | |
| GTC 347^{T} | | | | | | | | | | |
| Escherichia vulneris | | | | | | | | | | |
| ATCC 33821^{T} | | | | | | | | | | |
| Shigella dysenteriae | | | | | | | | | | |
| ATCC 13313^{T} | | | | | | | | | | |
| Shigella flexneri | | | | | | | | | | |
| ATCC 29903^{T} | | | | | | | | | | |
| Shigella sonnei | | | | | | | | | | |
| GTC 781^{T} | | | | | | | | | | |
| Salmonella bongori | | | | | | | | | | |
| BR 1859^{T} | | | | | | | | | | |
| Salmonella enterica | | | | | | | | | | |
| ssp arizonae | | | | | | | | | | |
| ATCC 13314^{T} | | | | | | | | | | |
| Brenneria alni | — | | | | | | | | | |
| DSM 11811^{T} | | | | | | | | | | |
| Brenneria salicis | 96.0 | — | | | | | | | | |
| DSM 30166^{T} | | | | | | | | | | |
| Dickeya chrysanthemi | 95.3 | 95.9 | — | | | | | | | |
| DSM 4610^{T} | | | | | | | | | | |
| Pectobacterium | 95.6 | 94.7 | 96.2 | — | | | | | | |
| carotovorum | | | | | | | | | | |
| DSM 30168^{T} | | | | | | | | | | |
| Edwardsiella hoshinae | 92.6 | 93.5 | 94.6 | 95.2 | — | | | | | |
| JCM1679^{T} | | | | | | | | | | |
| Edwardsiella ictaluri | 92.9 | 93.6 | 95.0 | 95.2 | 99.3 | — | | | | |
| JCM1680^{T} | | | | | | | | | | |
| Cedecea davisae | 94.1 | 94.3 | 95.1 | 96.6 | 95.6 | 95.5 | — | | | |
| DSM 4568^{T} | | | | | | | | | | |
| Cedecea neteri | 94.4 | 94.3 | 95.2 | 96.6 | 95.6 | 958 | 99.0 | — | | |
| GTC1717^{T} | | | | | | | | | | |
| Trabulsiella | 93.3 | 92.9 | 94.2 | 94.7 | 95.4 | 95.3 | 95.5 | 95.5 | — | |
| guamensis | | | | | | | | | | |
| ATCC 49490^{T} | | | | | | | | | | |
| Cronobacter sakazakii | 93.0 | 93.7 | 95.0 | 94.7 | 95.5 | 95.3 | 96.3 | 95.3 | 96.8 | — |
| ATCC 29544^{T} | | | | | | | | | | |

Table 5 is the gene sequence (SEQ ID NO: 1) for the 16S rRNA for SJCH-12.

TABLE 5
LOCUS 1083-08 1530 bp RNA RNA 23 JAN. 198
BASE COUNT 381 a 354 c 484 g 309 t 2 others

| | |
|---|---|
| 1 | TTTGATCCTG GCTCAGATTG AACGCTGGCG GCAGGCCTAA CACATGCAAG TCGAACGGTA |
| 61 | ACAGGAANCA GCTTGCTGNT TCGCTGACGA GTGGCGGACG GGTGAGTAAT GTCTGGGAAA |
| 121 | CTGCCTGATG GAGGGGGATA ACTACTGGAA ACGGTAGCTA ATACCGCATA ACGTCGCAAG |
| 181 | ACCAAAGAGG GGGACCTTCG GGCCTCTTGC CATCGGATGT GCCCAGATGG GATTAGCTAG |
| 241 | TAGGTGGGGT AAAGGCTCAC CTAGGCGACG ATCCCTAGCT GGTCTGAGAG GATGACCAGC |
| 301 | CACACTGGAA CTGAGACACG GTCCAGACTC CTACGGGAGG CAGCAGTGGG GAATATTGCA |
| 361 | CAATGGGCGC AAGCCTGATG CAGCCATGCC GCGTGTATGA AGAAGGCCTT CGGGTTGTAA |
| 421 | AGTACTTTCA GCGGGGAGGA AGGGAGTAAA GTTAATACCT TTGCTCATTG ACGTTACCCG |
| 481 | CAGAAGAAGC ACCGGCTAAC TCCGTGCCAG CAGCCGCGGT AATACGGAGG GTGCAAGCGT |
| 541 | TAATCGGAAT TACTGGGCGT AAAGCGCACG CAGGCGGTTT GTTAAGTCAG ATGTGAAATC |
| 601 | CCCGGGCTCA ACCTGGGAAC TGCATCTGAT ACTGGCAAGC TTGAGTCTCG TAGAGGGGGG |
| 661 | TAGAATTCCA GGTGTAGCGG TGAAATGCGT AGAGATCTGG AGGAATACCG GTGGCGAAGG |
| 721 | CGGCCCCCTG GACGAAGACT GACGCTCAGG TGCGAAAGCG TGGGGAGCAA ACAGGATTAG |
| 781 | ATACCCTGGT AGTCCACGCC GTAAACGATG TCGACTTGGA GGTTGTGCCC TTGAGGCGTG |
| 841 | GCTTCCGGAG CTAACGCGTT AAGTCGACCG CCTGGGGAGT ACGGCCGCAA GGTTAAAACT |
| 901 | CAAATGAATT GACGGGGGCC CGCACAAGCG GTGGAGCATG TGGTTTAATT CGATGCAACG |
| 961 | CGAAGAACCT TACCTGGTCT TGACATCCAC GGAAGTTTTC AGAGATGAGA ATGTGCCTTC |
| 1021 | GGGAACCGTG AGACAGGTGC TGCATGGCTG TCGTCAGCTC GTGTTGTGAA ATGTTGGGTT |
| 1081 | AAGTCCCGCA ACGAGCGCAA CCCTTATCCT TTGTTGCCAG CGGTCCGGCC GGGAACTCAA |
| 1141 | AGGAGACTGC CAGTGATAAA CTGGAGGAAG GTGGGGATGA CGTCAAGTCA TCATGGCCCT |
| 1201 | TACGACCAGG GCTACACACG TGCTACAATG GCGCATACAA AGAGAAGCGA CCTCGCGAGA |
| 1261 | GCAAGCGGAC CTCATAAAGT GCGTCGTAGT CCGGATTGGA GTCTGCAACT CGACTCCATG |
| 1321 | AAGTCGGAAT CGCTAGTAAT CGTGGATCAG AATGCCACGG TGAATACGTT CCCGGGCCTT |
| 1381 | GTACACACCG CCCGTCACAC CATGGGAGTG GGTTGCAAAA GAAGTAGGTA GCTTAACCTT |
| 1441 | CGGGAGGGCG CTTACCACTT TGTGATTCAT GACTGGGGTG AAGTCGTAAC AAGGTAACCG |
| 1501 | TAGGGGAACC TGCGGCTGGA TCACCTCCTT |

### REFERENCES

Acebal, C., Canedo, L. M. Puentes, J. L. Fl, Baz, J. P., Romero, B., Da La Calle, and Rodrigues, M. 1999. Agrochelin, a new cytotoxic antiviotice from a marine agrobacterium. Isolation, physiochemical poperties and biological activity. J. Antibiotic. 52:983-987.
Biabani, M. A. F., Laatsch, H., Helmke, E., and Weyland, H. 1997. d-Indomycinone: class of antibiotics isolated from marine Streptomyces sp. J. Antibiot. 50: 874-877.
Gauthier, G., Gauthier, M. and Christen, R., 1995. Phylogenetic analysis of the genera and Moritella using genes coding for small-subunit rRNA sequence and division of the genera, Alteromonas (emended) and Pseudoalteromonas gen. nov., and proposal of twel Int. J. Syst. Bacteriol. 45: 755-761.
Imamura, N., Nishijima, M. Takadera, T., Adachi, K., Sakai, M. and Sano, H. 1997. pelagiomicins produced by a new marine bacterium Pelagiobacter variabilis. J. Antibiot.
Pusecker, K., Laatsch, H., Helmke, E., and Weyland, H. 1997. dihydrophencomycin phenazine derivative from a marine Streptomycete. J. Antibiot. 50: 479-483.
Isnansetyo, A. and Kamei, Y. 2003. MC21-A, a Bactericidal Antibiotic Produced by a new Marine Bacterium Pseudoalteromonas phenolica sp. nov. O-BC30T, against Methicillin Resistant Staphylococcus aureus. Antimicrob. Agents chemother. February: 47(2) 48-488.
Usdin, S., BioCentury, Jun. 26, 2006.
Chemistry & Engineering News, Jan. 28, 2008, _{P.} 57.

What is claimed is:

1. A composition comprising an antibacterial polypeptide produced by an isolated bacterial cell culture having Deutsche Sammlung von Mikroorganismen und Zellkulturen (DSMZ) accession number DSM 21971, the polypeptide having a molecular weight of about 5 kDa and a pI of about 6.8-7.2 when isolated by polyacrylamide gel electrophoresis (PAGE).

2. The composition of claim 1 wherein the antibacterial polypeptide exhibits antibiotic activity against Staphylococcus aureus, Methicillin Resistant Staphylococcus aureus, Escherichia coli, Enterobacter aerogenes, Proteus vulgaris, Klebsiella pneumoniae, Profidencia alcalifaciens, Klebsiella oxytoca, Acinetobacter baumani, Serratia liquefaciens, and Pseudomonas aeruginosa.

3. The composition of claim 1 which is comprised within a pharmaceutically acceptable carrier.

4. A method for treatment or prevention of a bacterial infection in a mammal in need thereof, comprising administering to said mammal an effective amount of the composition of claim 3.

5. The method of claim 3 wherein the bacterial infection is Staphylococcus aureus, Methicillin Resistant Staphylococcus aureus, Escherichia coli, Enterobacter aerogenes, Proteus vulgaris, Klebsiella pneumoniae, Profidencia alcalifaciens, Klebsiella oxytoca, Acinetobacter baumani, Serratia liquefaciens, and Pseudomonas aeruginosa.

6. The method of claim 4 wherein the bacterial infection is from Methicillin Resistant Staphylococcus aureus MRSA).

7. A kit comprising an isolated bacterial cell culture having Deutsche Sammlung von Mikroorganismen und Zellkulturen (DSMZ) accession number DSM 21971 and a protein isolated from said culture which has a molecular weight of about 5 kDa, a pI of about 6.8-7.2 when isolated by polyacrylamide gel electrophoresis (PAGE), and antibiotic activity against Staphylococcus aureus, Methicillin Resistant Staphylococcus aureus, Escherichia col, Enterobacter aerogenes, Proteus vulgaris, Klebsiella pneumoniae, Profidencia alcalifaciens, Klebsiella oxytoca, Acinetobacter baumannii, Serratia liquefaciens, and Pseudomonas aeruginosa.

8. The kit of claim 7 wherein the protein isolated from said culture is thermostable at temperatures within a range of about 4° C. up to at least 37° C.

9. The kit of claim 7 wherein the protein is isolated from said bacterial cell culture under conditions that allow for expression in liquid media or on agar.
